(19) United States
(12) Patent Application Publication
(10) Pub. No.: US 2015/0359590 A1
(43) Pub. Date: Dec. 17, 2015

(54) Implantable Catheter-Delivered Neuromodulation Devices and Related Devices, Systems, and Methods

(71) Applicant: Medtronic Ardian Luxembourg S.a.r.l., Luxembourg (LU)

(72) Inventors: Barry O'Connell, Galway (IE); Cormac Delaney, Galway (IE); Angela Duffy, Galway (IE); Eoghan Twohig, Galway (IE)

(21) Appl. No.: 14/306,997

(22) Filed: Jun. 17, 2014

(51) Int. Cl. A61B 18/14

(52) U.S. Cl. CPC A61B 18/1492; A61B 2018/0022

(57) ABSTRACT

An example of an implantable neuromodulation device includes a bioabsorbable electrode and an elongate bioabsorbable support structure carrying the electrode. The support structure is configured to expand in a direction perpendicular to its length so as to move the electrode into contact with a wall of a naturally occurring lumen of a human patient. The electrode is electrically activatable to modulate a nerve within tissue at or otherwise proximate to the wall of the lumen. An example of a neuromodulation method using the neuromodulation device includes locating the neuromodulation device at a treatment site within the lumen and deploying the neuromodulation device into an expanded treatment state at the treatment site. The method further includes reducing obstruction of blood flow through the lumen after deploying the neuromodulation device and then wirelessly energizing the electrode from an extracorporeal energy source.

### TECHNICAL FIELD

The present technology is related to neuromodulation devices, such as implantable catheter-delivered neuromodulation devices.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states. Excessive activation of the renal SNS, in particular, has been identified experimentally and in humans as a likely contributor to the complex pathophysiologies of hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function are considerably stimulated in disease states characterized by heightened sympathetic tone. For example, reduced renal blood flow and glomerular filtration rate as a result of renal sympathetic efferent stimulation is likely a cornerstone of the loss of renal function in cardio-renal syndrome. Pharmacologic strategies to mitigate adverse consequences of renal sympathetic stimulation often include the use of centrally-acting sympatholytic drugs, beta blockers, angiotensin-converting enzyme inhibitors, and/or diuretics. These and other pharmacologic strategies, however, tend to have significant limitations including limited efficacy, compliance issues, and undesirable side effects.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present technology. For ease of reference, throughout this disclosure identical reference numbers may be used to identify identical or at least generally similar or analogous components or features.

FIG. 1 is a perspective view illustrating a neuromodulation system in accordance with an embodiment of the present technology. As shown in FIG. 1, the system can include a first catheter, a second catheter, and an extracorporeal accessory. The first catheter can include a shaft, an expandable structure, and a neuromodulation device.

FIG. 2 is a flow chart illustrating a neuromodulation method in accordance with an embodiment of the present technology.

FIGS. 3-5 are partially cross-sectional side views of the first catheter and an associated sheath while the sheath is retracted from a distal end portion of the first catheter and the neuromodulation device is at a treatment site within a renal artery.

In FIG. 3, the expandable structure is not expanded and the neuromodulation device is not implanted at the treatment site.

In FIG. 4, the expandable structure is expanded and the neuromodulation device is implanted at the treatment site.

In FIG. 5, the expandable structure is retracted and the neuromodulation device remains implanted at the treatment site. As shown in FIG. 3, the neuromodulation device can include an energy-delivery element.

FIG. 6 is a partially cross-sectional side view of the neuromodulation device implanted at the treatment site while other portions of the first catheter are removed from the treatment site.

FIG. 7 is an enlarged cross-sectional end view taken along the line 7-7 in FIG. 6.

FIG. 8 is an enlarged partially cross-sectional side view of the neuromodulation device and the extracorporeal accessory. As shown in FIG. 8, the extracorporeal accessory can include an extracorporeal energy source. In FIG. 8, the neuromodulation device is implanted at the treatment site and the extracorporeal energy source is wirelessly energizing the energy-delivery element.

FIG. 9 is an enlarged partially cross-sectional side view of the neuromodulation device and the first catheter. As shown in FIG. 9, the first catheter can include a first intracorporeal energy source. In FIG. 9, the neuromodulation device is implanted at the treatment site and the first intracorporeal energy source is wirelessly energizing the energy-delivery element.

FIG. 10 is an enlarged partially cross-sectional side view of the neuromodulation device and the second catheter. As shown in FIG. 10, the second catheter can include a second intracorporeal energy source. In FIG. 10, the neuromodulation device is implanted at the treatment site and the second intracorporeal energy source is wirelessly energizing the energy-delivery element.

FIG. 11 is an enlarged partially cross-sectional side view of the neuromodulation device implanted at the treatment site while the neuromodulation device is bioabsorbing.

FIG. 12 is a flattened plan view of a neuromodulation device in accordance with another embodiment of the present technology.

FIG. 13 is a flattened plan view of a neuromodulation device in accordance with yet another embodiment of the present technology.

FIG. 14 is an enlargement of a designated portion of FIG. 13.

### DETAILED DESCRIPTION

The present technology is related to catheter-delivered devices, such as implantable, catheter-delivered neuromodulation devices. A neuromodulation device of a catheter in accordance with a particular embodiment of the present technology is configured to be implanted at a treatment site within a naturally occurring lumen of a human patient. The neuromodulation device can include an energy-delivery element (e.g., an electrode or an ultrasound transducer) configured to modulate a nerve within tissue at or otherwise proximate to a wall of the lumen. When the neuromodulation device is initially implanted at the treatment site, blood flow through the treatment site may be at least partially obstructed. The neuromodulation device can be configured to remain implanted while obstruction of blood flow through the treatment site is reduced, such as by withdrawing a remaining portion of the catheter from the treatment site. Thereafter, an extracorporeal energy source and/or an intracorporeal energy source can be used to wirelessly energize the energy-delivery element. During neuromodulation, blood flow through the treatment site can be relatively unobstructed. This can be useful, for example, to facilitate heat dissipation from the wall of the lumen and/or to reduce or eliminate the possibility of ischemia downstream from the treatment site. Additional and/or alternative advantages of devices, systems, and methods in accordance with embodiments of the present technology are also possible.

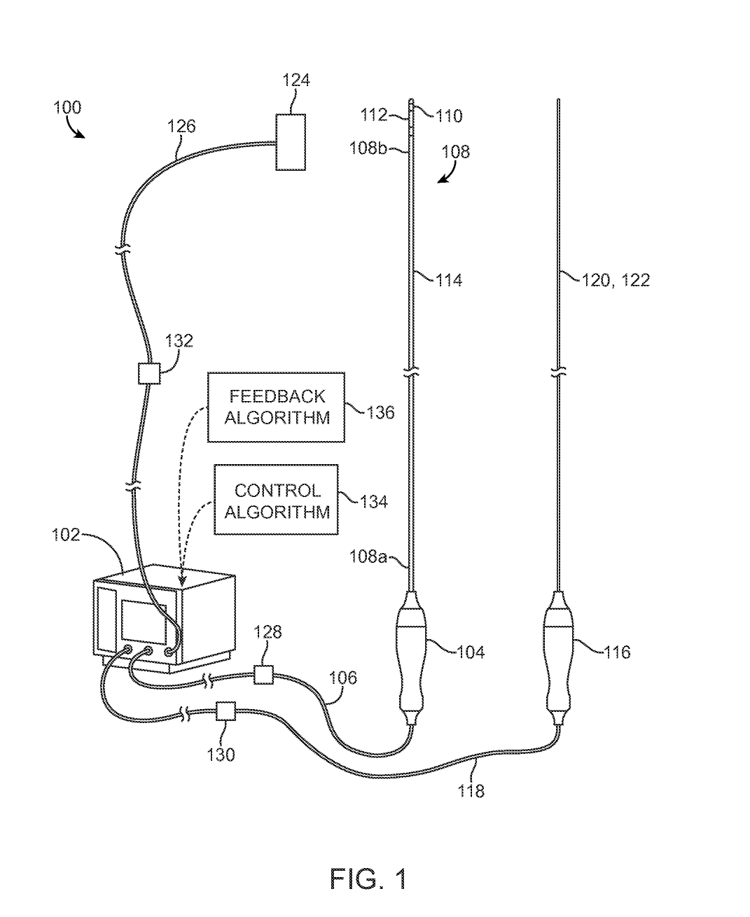

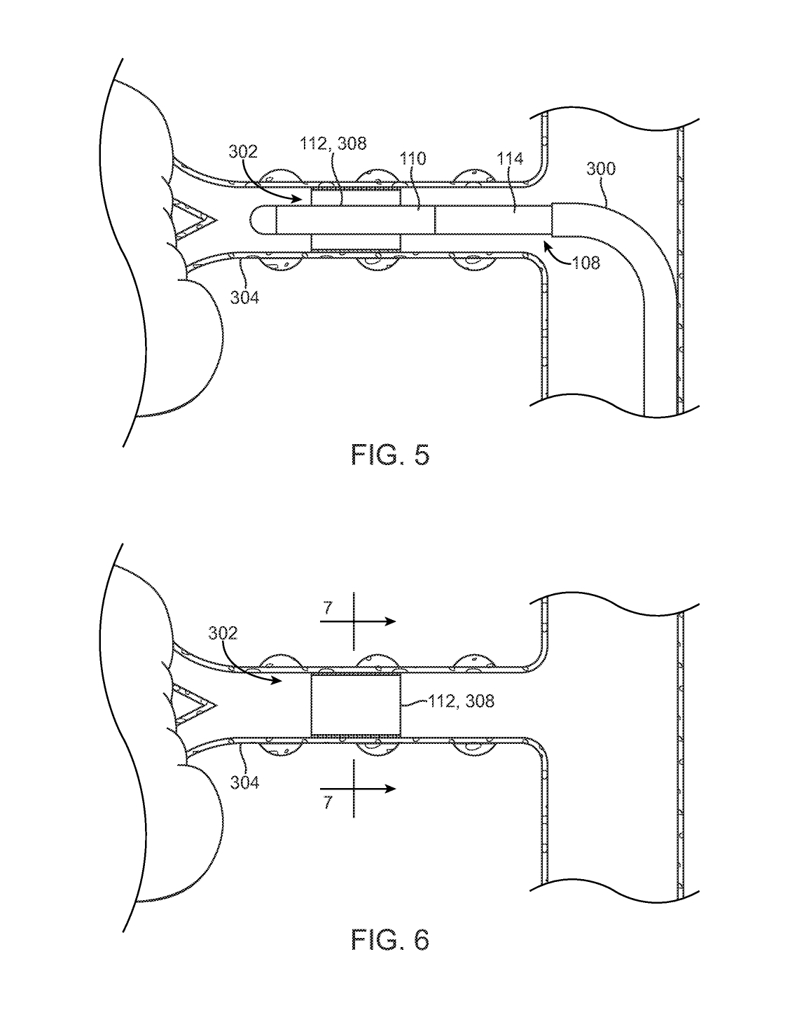

Specific details of devices, systems, and methods in accordance with several embodiments of the present technology are disclosed herein with reference to FIGS. 1-14. Although the devices, systems, and methods may be disclosed herein primarily or entirely with respect to intravascular renal neuromodulation, other applications in addition to those disclosed herein are within the scope of the present technology. For example, devices, systems, and methods in accordance with at least some embodiments of the present technology may be useful for neuromodulation within one or more non-vessel body lumens, for extravascular neuromodulation, for non-renal neuromodulation, and/or for use in therapies other than neuromodulation. Furthermore, it should be understood, in general, that other devices, systems, and methods in addition to those disclosed herein are within the scope of the present technology. For example, devices, systems, and methods in accordance with embodiments of the present technology can have different and/or additional configurations, components, and procedures than those disclosed herein. Moreover, a person of ordinary skill in the art will understand that devices, systems, and methods in accordance with embodiments of the present technology can be without one or more of the configurations, components, and/or procedures disclosed herein without deviating from the present technology.

### Selected Examples of Neuromodulation Catheters and Associated Technology

FIG. 1 is a perspective view illustrating a neuromodulation system 100 configured in accordance with an embodiment of the present technology. The system 100 can include a console 102, a first handle 104, and a first cable 106 extending therebetween. The system 100 can further include a first catheter 108 operably connected to the first handle 104. The first catheter 108 can have a proximal end portion 108a and a distal end portion 108b. At its distal end portion 108b, the first catheter 108 can include an elongate expandable structure 110 and an elongate neuromodulation device 112 extending circumferentially around the expandable structure 110. The first catheter 108 can further include a first shaft 114 extending between the expandable structure 110 and the first handle 104. The first shaft 114 can be configured to locate the neuromodulation device 112 at a treatment site within a naturally occurring lumen of a human patient, such as a suitable blood vessel, duct, airway, or other naturally occurring lumen at any suitable branching level. Once located, the neuromodulation device 112 can be configured to provide or support a neuromodulation treatment.

The system 100 can further include a second handle 116, a second cable 118 extending between the console 102 and the second handle 116, and a second catheter 120 operably connected to the second handle 116. The second catheter 120 can include a second shaft 122 extending distally from the second handle 116. The second shaft 122 can be configured to achieve an operable position for intracorporeal energy delivery to the neuromodulation device 112 while the neuromodulation device 112 is implanted at a treatment site and the first catheter 108 is removed from the treatment site. The system 100 can further include an extracorporeal accessory 124 and a third cable 126 extending between the console 102 and the extracorporeal accessory 124. The extracorporeal accessory 124 can be configured for extracorporeal energy delivery to the neuromodulation device 112 while the neuromodulation device 112 is implanted at a treatment site. The neuromodulation device 112 can be configured to receive energy from the first catheter 108 with or without a direct connection to the first catheter 108, to receive energy from the second catheter 120 with or without a direct connection to the second catheter 120, wirelessly from the extracorporeal accessory 124, and/or in another suitable manner.

The console 102 can be configured to control, monitor, supply energy to, and/or otherwise support operation of the first catheter 108, the second catheter 120, and the extracorporeal accessory 124. Alternatively, the first handle 104 and the first catheter 108 in combination and/or the second handle 116 and the second catheter 120 in combination can be self-contained or otherwise configured for operation without connection to the console 102. Alternatively or in addition, the first catheter 108 alone, the second catheter 120 alone, and/or the extracorporeal accessory 124 alone can be self-contained or otherwise configured for operation without connection to the console 102. When present, the console 102 can be a generator system including an energy generator (not shown) configured to generate a selected form and/or magnitude of energy for delivery to tissue at a treatment site via the neuromodulation device 112. In at least some cases, this is in conjunction with another portion of the first catheter 108, in conjunction with the second catheter 120, and/or in conjunction with the extracorporeal accessory 124. The console 102 can have different configurations depending on the treatment modality of the neuromodulation device 112. For example, when the neuromodulation device 112 is configured for electrode-based, heat-element-based, or transducer-based treatment, the console 102 can include an energy generator configured to generate radio frequency (RF) energy (e.g., monopolar and/or bipolar RF energy), pulsed electrical energy, microwave energy, optical energy, ultrasound energy (e.g., high-intensity focused ultrasound energy), direct heat, radiation (e.g., infrared, visible, and/or gamma radiation), and/or one or more other suitable types of energy.

The system 100 can include a first control device 128, a second control device 130, and a third control device 132 respectively disposed along the first cable 106, the second cable 118, and the third cable 126. Alternatively, the first control device 128, the second control device 130, and the third control device 132 can be respectively incorporated into the first handle 104, the second handle 116, and the extracorporeal accessory 124 or have other suitable positions within the system 100. The first control device 128 can be configured to control (e.g., to electrically control) operation of the first catheter 108 directly and/or via the console 102. For example, the first control device 128 can be configured to control expansion and retraction of the expandable structure 110. The second control device 130 and the third control device 132 can be configured, respectively, to control (e.g., to electrically control) operation of the second catheter 120 and the extracorporeal accessory 124 directly and/or via the console 102. In at least some embodiments, the system 100 is configured to be integrated (e.g., wirelessly integrated) into a higher-level system, such as an overall control and/or monitoring system of an operating room.

When the system 100 is in use, an operator can use the first control device 128, the second control device 130, and/or the third control device 132 to provide instructions to the console 102, such as to initiate or to terminate a neuromodulation treatment. In addition to being configured to execute such instructions, the console 102 can be configured to execute an automated control algorithm 134. Furthermore, the console 102 can be configured to provide information to an operator before, during, and/or after a neuromodulation procedure via a feedback algorithm 136. Feedback from the feedback algorithm 136 can be audible, visual, haptic, or have another suitable form. The feedback can be based on output from a monitoring system (not shown). For example, such a monitoring system can include a monitoring device (e.g., a sensor) configured to measure a condition at a treatment site (e.g., a temperature of tissue being treated), a systemic condition (e.g., a patient vital sign), or another condition germane to the treatment, health, and/or safety of a patient. The monitoring device can be integrated into the first catheter 108 and/or integrated into the second catheter 120. Alternatively, the monitoring device can be separate from the first and second catheters 108, 120 and/or separate from the system 100.

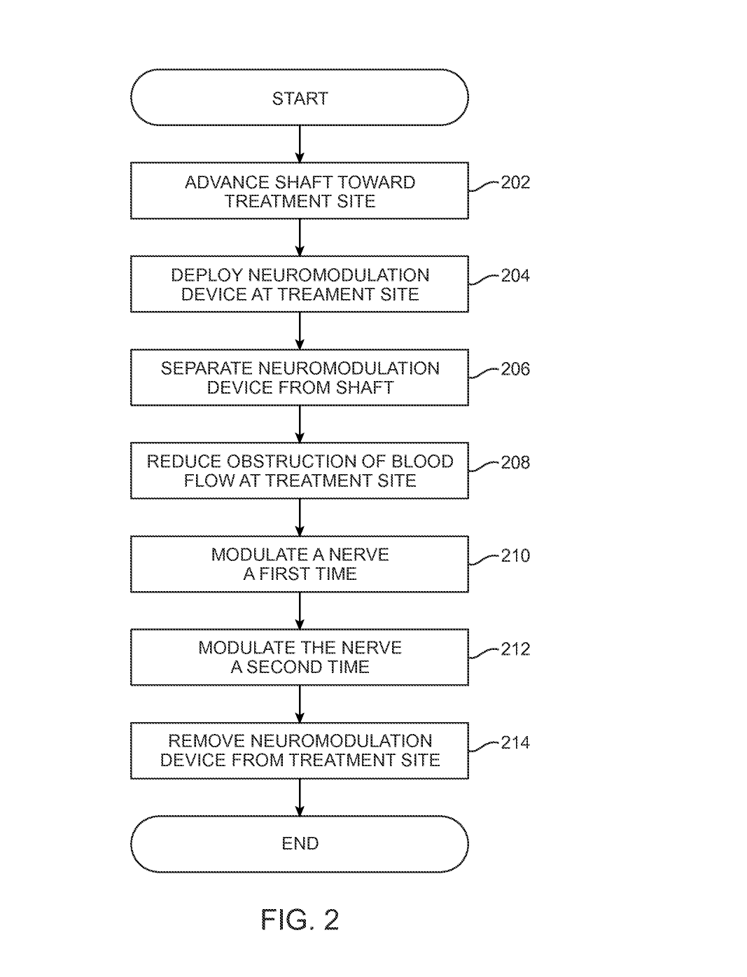

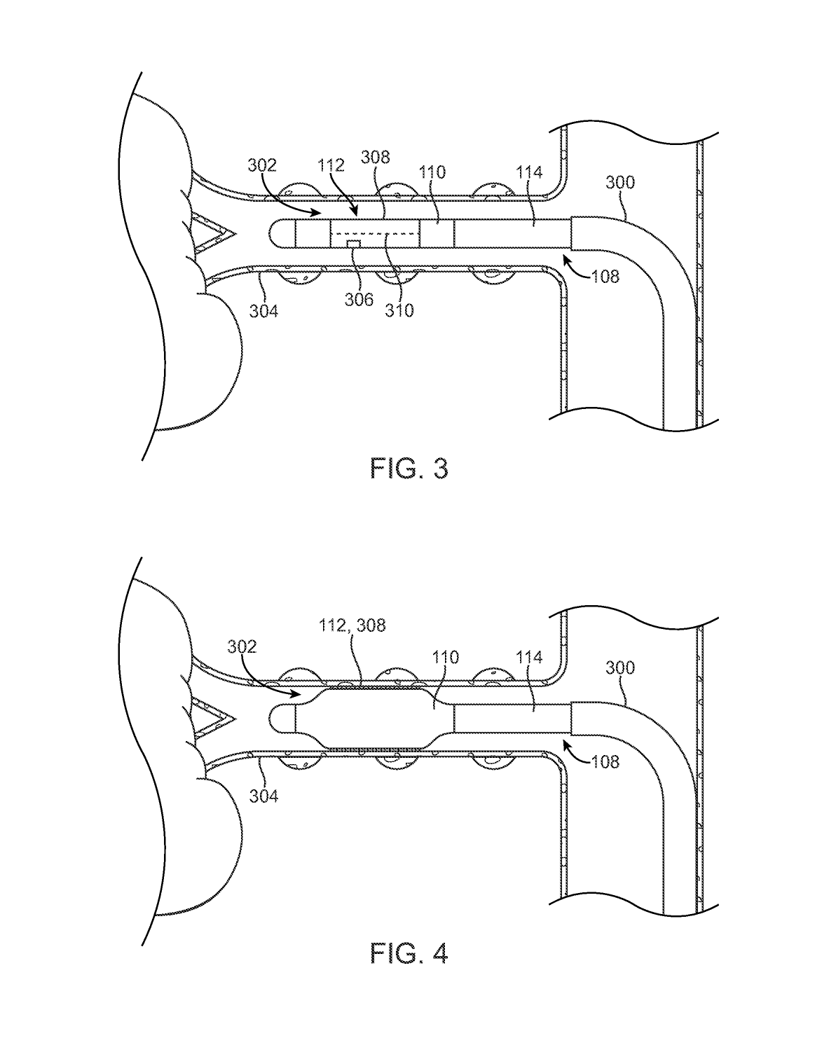

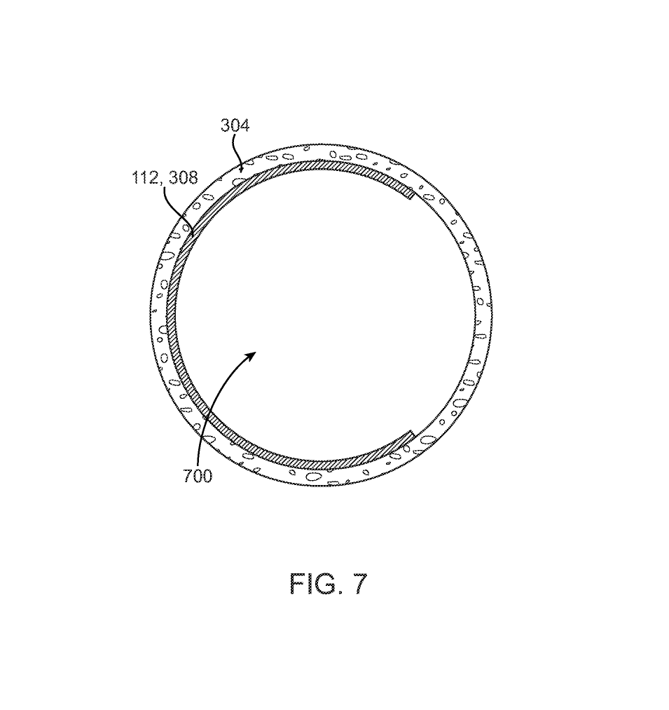

FIG. 2 is a flow chart illustrating a neuromodulation method 200 in accordance with an embodiment of the present technology. FIGS. 3-5 are partially cross-sectional side views of the first catheter 108 and an associated sheath 300 while the sheath 300 is retracted from the distal end portion 108b of the first catheter 108 and the neuromodulation device 112 is at a treatment site 302 within a body lumen 304. FIG. 6 is a partially cross-sectional side view of the neuromodulation device 112 implanted at the treatment site 302 while other portions of the first catheter 108 are removed from the treatment site 302. FIG. 7 is an enlarged cross-sectional end view taken along the line 7-7 in FIG. 6. With reference to FIGS. 2-7 together, the method 200 can include advancing the first shaft 114 toward the treatment site 302 while the neuromodulation device 112 is in a low-profile delivery state (block 202). Once the neuromodulation device 112 is at the treatment site 302, the method 200 can include deploying the neuromodulation device 112 into an expanded treatment state (block 204). The neuromodulation device 112 can include an energy-delivery element 306 (e.g., an electrode, a direct heat element, or an ultrasound transducer) (FIG. 3) and an elongate support structure 308 carrying the energy-delivery element 306. The support structure 308 can be configured to expand in a direction perpendicular to its length so as to move the energy-delivery element 306 into contact with a wall of the body lumen 304.

In the illustrated embodiment, the support structure 308 is tubular and includes a seam 310 extending parallel to its length. The support structure 308 can be resiliently biased toward expanding radially outward away from the first shaft 114. For example, the support structure 308 can have a tendency to uncurl into a relatively flat form when unconstrained. At the seam 310, the support structure 308 can be perforated, thinned, or otherwise weakened to create a preferential breaking axis. In this manner or in another suitable manner, the support structure 308 can be configured to break apart predictably (e.g., at the seam 310) when it expands. The expandable structure 110 can releasably carry the neuromodulation device 112 and can be configured to expand in transverse cross-sectional area so as to cause the support structure 308 to break apart at the seam 310. The expandable structure 110 can be a balloon (e.g., a zero-fold balloon), a resilient sponge, a resilient polymeric mass, or have another suitable form. In at least some cases, breaking apart at the seam 310 reduces constraint on the support structure 308 and thereby allows the support structure 308 to expand resiliently outward toward the wall of the body lumen 304. During or after deployment of the neuromodulation device 112, the method 200 can include separating (e.g., completely separating) the neuromodulation device 112 from the first shaft 114 (block 206). In at least some cases, this occurs as the expandable structure 110 is retracted inwardly away from the neuromodulation device 112 while the neuromodulation device 112 remains implanted. The neuromodulation device 112 can remain implanted, for example, due to an outward force resiliently exerted by the support structure 308 against the wall of the body lumen 304.

In another embodiment, an alternative neuromodulation device (not shown) similar to the neuromodulation device 112 is configured to be deployed without the expandable structure 110. For example, the alternative neuromodulation device can be configured to be deployed as it exits the sheath 300. The sheath 300 can constrain the alternative neuromodulation device in a delivery state while the first shaft 114 advances toward the treatment site 302. The alternative neuromodulation device can be deployed from within the sheath 300 so as to allow the alternative neuromodulation device to resiliently expand into a treatment state. In another embodiment, an alternative neuromodulation device (also not shown) is configured to be magnetically coupled to an alternative expandable structure to constrain the alternative neuromodulation device in a delivery state. The alternative neuromodulation device can be magnetically uncoupled from the alternative expandable structure at the treatment site 302 to cause the alternative neuromodulation device to expand into a treatment state. In another embodiment, an alternative neuromodulation device (also not shown) has the structure of an expandable stent used in coronary or other clinical contexts. Other variations of the neuromodulation device 112 are also possible.

With reference again to FIGS. 2-7 together, while the neuromodulation device 112 is being deployed at the treatment site 302, the first shaft 114 and the expandable structure 110 may at least partially obstruct blood flow through the body lumen 304 at the treatment site 302. As discussed above, reduced blood flow during a neuromodulation procedure can be disadvantageous. Blood flow tends to facilitate heat dissipation that reduces or eliminates the possibility of causing collateral thermal damage to a wall of the body lumen 304 during a neuromodulation procedure. Furthermore, when blood flow is significantly obstructed during a neuromodulation procedure, the clinically acceptable duration of the procedure may be limited so as to prevent the onset of ischemia downstream from the treatment site 302.

In contrast to at least some alternatives, the neuromodulation device 112 may allow blood flow through the treatment site 302 to be at least partially restored (e.g., at least 25% restored, at least 50% restored, or at least 90% restored) while the neuromodulation device 112 remains deployed at the treatment site 302. For example, the method 200 can include reducing obstruction of blood flow through the body lumen 304 at the treatment site 302 (block 208) (e.g., reducing obstruction by at least 25%, by at least 50%, or by at least 90%) after deploying the neuromodulation device 112 and while the neuromodulation device 112 remains in the treatment state at the treatment site 302. In at least some cases, decreasing the transverse cross-sectional area of the expandable structure 110 within an interior region 700 (FIG. 7) defined by the neuromodulation device 112 may cause at least some reduced obstruction of blood flow through the body lumen 304. Alternatively or in addition, withdrawing the first shaft 114 and the expandable structure 110 from the interior region 700 may cause at least some reduced obstruction of blood flow through the body lumen 304. Furthermore, withdrawing the sheath 300 away from the treatment site 302 may cause at least some reduced obstruction of blood flow through the body lumen 304. Other manners of reducing obstruction of blood flow through the body lumen 304 are also possible.

The method 200 can further include modulating a nerve within tissue at or otherwise proximate to a wall of the body lumen 304 at the treatment site 302 a first time (block 210) and then a second time (block 212). Alternatively, the method 200 can include modulating the nerve only once. Modulating the nerve can include delivering energy to the tissue via the energy-delivery element 306 while obstruction of blood flow through the body lumen 304 is reduced. Thus, advantageous blood flow through the body lumen 304 at the treatment site 302 can occur during neuromodulation. This can allow a greater amount of energy to be delivered to the tissue, allow for a longer treatment period, and/or have other advantages relative to at least some alternatives. These advantages may allow the neuromodulation device 112 to be used in anatomy that may otherwise be inaccessible to neuromodulation. For example, although a relatively large renal artery is shown as the body lumen 304 in FIGS. 3-6, the body lumen 304 can alternatively be relatively small. In some cases, the method 200 is carried out at a treatment site 302 within a body lumen 304 having a diameter less than 2 millimeters, such as less than 1.5 millimeter or even less than 1 millimeter at the treatment site 302. Neuromodulation within relatively small body lumens 304 tends to be challenging due, at least in part, to the relatively high susceptibility of such body lumens 304 to thermal damage.

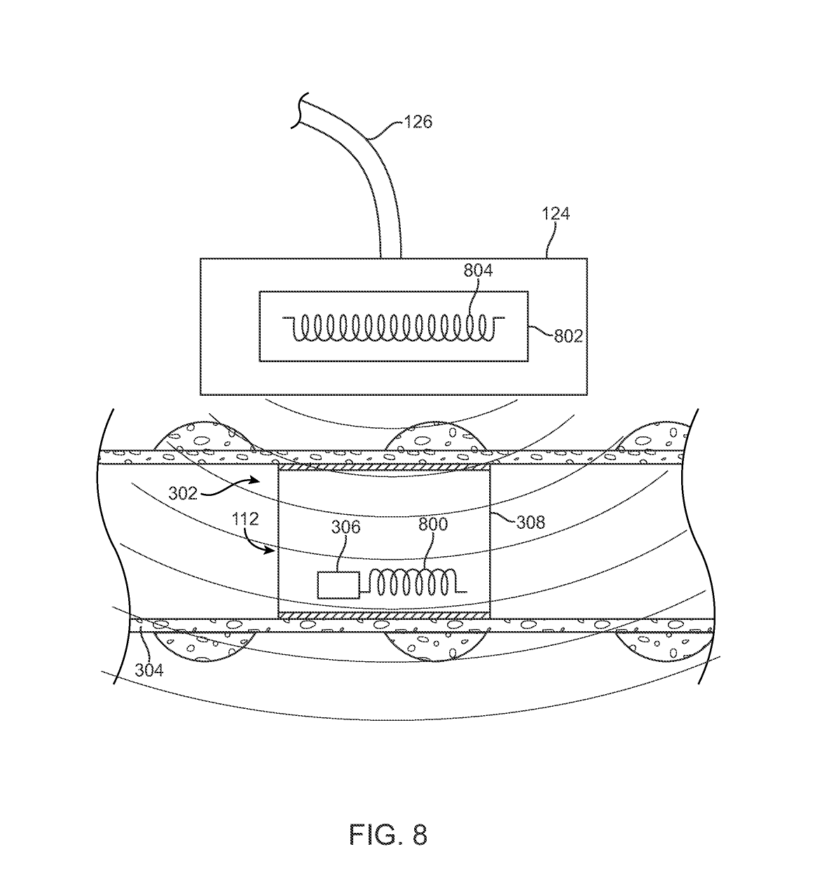

The energy-delivery element 306 can be configured to be energized (e.g., electrically energized) intracorporeally and/or extracorporeally. FIG. 8 illustrates an example of extracorporeally energizing the energy-delivery element 306. As shown in FIG. 8, the neuromodulation device 112 can include an inductor 800 (e.g., an induction coil) operably connected to the energy-delivery element 306. The extracorporeal accessory 124 can include an extracorporeal energy source 802 that has another inductor 804 (e.g., another induction coil) and is configured to wirelessly energize the energy-delivery element 306 in conjunction with the inductor 800. The inductors 800, 804 can be resonance coupled to facilitate wireless energy transmission from the extracorporeal energy source 802 to the energy-delivery element 306.

During a neuromodulation procedure, the extracorporeal accessory 124 can be held against a portion of a patient's skin close to an internal treatment site 302 at which the neuromodulation device 112 is implanted. The extracorporeal accessory 124 can receive electrical power from the console 102 (FIG. 1) via the third cable 126. This electrical power can be used to energize the inductor 804. Energizing the inductor 804 while the inductor 804 is in relatively close proximity to the inductor 800 (e.g., within 20 cm, within 10 cm, or within another suitable distance) can cause the inductor 800 to generate electrical power by induction for immediate and/or delayed delivery to the energy-delivery element 306. In this or another suitable manner of wireless energy transmission, the implanted neuromodulation device 112 can be operably independent of other portions of the first catheter 108. Thus, the other portions of the first catheter 108 can be withdrawn from the interior region 700 before or during neuromodulation such that blood flow through the body lumen 304 at the treatment site 302 via the interior region 700 is relatively unimpeded.

In at least some cases, the interior region 700 need not be entirely vacant to allow for sufficient blood flow through the body lumen 304 at the treatment site 302. Neuromodulation, therefore, can be carried out while the first shaft 114 and the expandable structure 110 and/or one or more other structures that collectively occupy only a portion of a transverse cross-sectional area of the interior region 700 remain within the interior region 700. In these and other cases, energy can be provided to the energy-delivery element 306 intracorporeally via a wired or wireless connection. When a wired connection is used, the neuromodulation device 112, after deployment, can be tethered to other portions of the first catheter 108 so that the other portions of the first catheter 108 can be withdrawn from the treatment site 302. Intracorporeal energy delivery to the energy-delivery element 306 can be useful, for example, when a patient's anatomy prevents location of the extracorporeal accessory 124 in sufficiently close proximity to the inductor 800 to allow for extracorporeal energy delivery to the energy-delivery element 306.

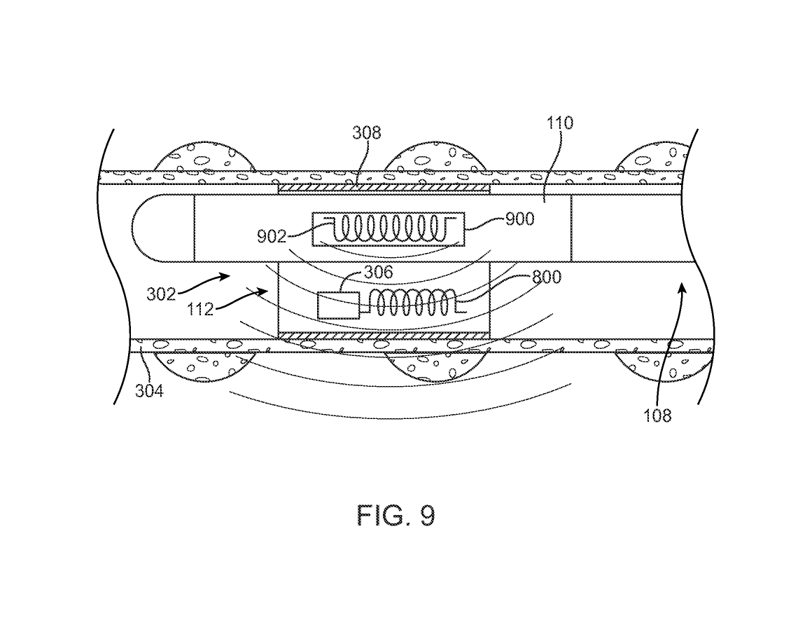

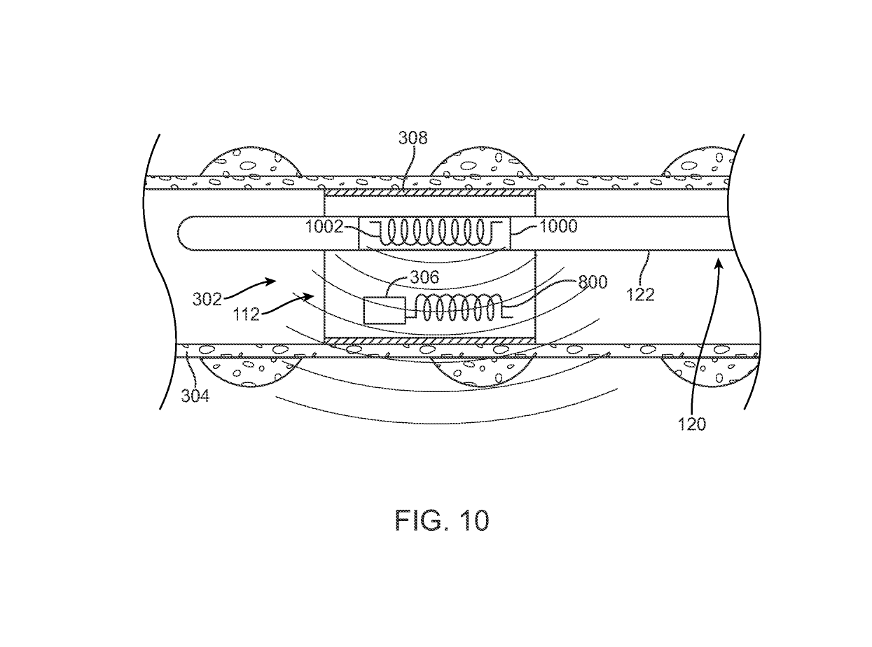

FIGS. 9 and 10 illustrate respective examples of intracorporeally energizing the energy-delivery element 306. As shown in FIG. 9, the first catheter 108 can include an intracorporeal energy source 900 having an inductor 902 (e.g., an induction coil) configured to wirelessly energize the energy-delivery element 306 in conjunction with the inductor 800. Thus, in a particular embodiment, the first catheter 108 is used for both deployment of the neuromodulation device 112 using the expandable structure 110 and wireless energy transmission to the neuromodulation device 112 using the intracorporeal energy source 900. The intracorporeal energy source 900 can be disposed within the expandable structure 110, at a surface of the expandable structure 110, or at another suitable position within the first catheter 108. In other embodiments, the first catheter 108 can be used for deployment of the neuromodulation device 112 only and the extracorporeal accessory 124, the second catheter 120, or another suitable component of the system 100 can be used for wired or wireless energy transmission to the neuromodulation device 112. As shown in FIG. 10, the second catheter 120 can include an intracorporeal energy source 1000 having an inductor 1002 (e.g., an induction coil) configured to wirelessly energize the energy-delivery element 306 in conjunction with the inductor 800. Without needing to carry the expandable structure 110 or any other deployment structures, the second catheter 120 can be narrower than the first catheter 108. This can facilitate greater blood flow through the interior region 700 when the second catheter 120 is positioned within the interior region 700 during a neuromodulation treatment.

When the neuromodulation device 112 is configured to execute a neuromodulation treatment without the presence of the first catheter 108, additional neuromodulation treatments following an initial neuromodulation treatment may be relatively convenient. For example, with reference again to FIG. 2, modulating the nerve a second time can occur at least 30 minutes, at least 1 hour, at least 24 hours, or after any other suitable period of time after modulating the nerve the first time. The neuromodulation device 112 can remain in the treatment state at the treatment site 302 for an interim between an initial and a follow-up neuromodulation treatment so that re-catheterization for the follow-up procedure is not necessary. Thus, it may be possible to perform one or more follow-up neuromodulation treatments on an out-patient basis as needed until a desired clinical outcome (e.g., blood pressure reduction) is achieved. For example, if a desired clinical outcome is not achieved several days, weeks, or months after an initial neuromodulation treatment, a follow-up neuromodulation treatment can be performed. This can be repeated until the desired clinical outcome is achieved. In a particular example, a first follow-up neuromodulation treatment occurs 30 days after an initial neuromodulation treatment, a second follow-up neuromodulation treatment occurs 60 days after an initial neuromodulation treatment, and/or a third follow-up neuromodulation treatment occurs 90 days after an initial neuromodulation treatment.

With reference again to FIG. 2, after one or more neuromodulation treatments using the neuromodulation device 112, the neuromodulation device 112 can be removed from the treatment site 302 (block 214). In some embodiments, this includes recovering the neuromodulation device 112. For example, the inductor 902 can be used as an electromagnet when the expandable structure 110 is expanded within the interior region 700 so as to cause the neuromodulation device 112 to magnetically attach to the expandable structure 110. The expandable structure 110 can then be reduced in transverse cross-sectional area to draw the neuromodulation device 112 back into a delivery state for removal from the treatment site 302. In other embodiments, the neuromodulation device 112 can be secured to the expandable structure 110 in another suitable manner. In still other embodiments, the neuromodulation device 112 can be recovered using the second catheter 120 with the inductor 902 acting as an electromagnet. In still other embodiments, a dedicated recovery catheter (not shown) including an electromagnet, a permanent magnet, or another suitable coupling mechanism can be used to recover the neuromodulation device 112.

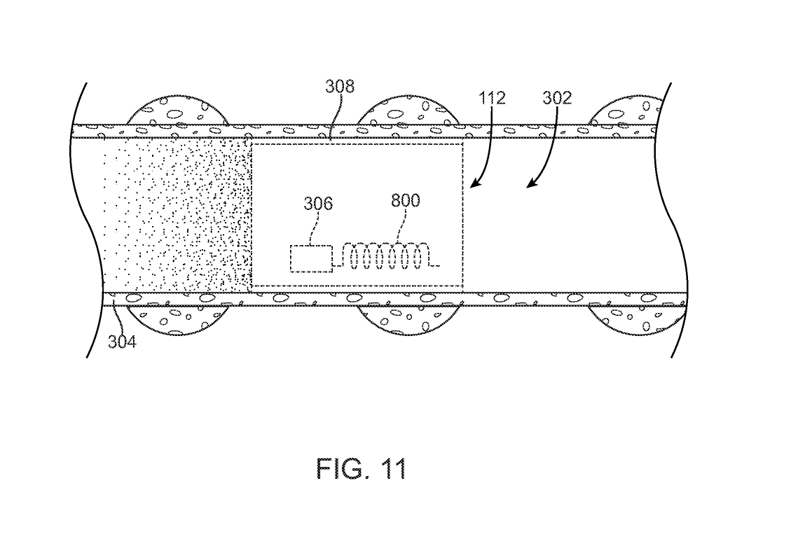

Removing the neuromodulation device 112 from the treatment site 302 need not include recovering the neuromodulation device 112. In some embodiments, the neuromodulation device 112 is bioabsorbable and removing the neuromodulation device 112 from the treatment site 302 includes disintegrating the neuromodulation device 112 at the treatment site 302. For example, FIG. 11 is an enlarged partially cross-sectional side view of the neuromodulation device 112 implanted at the treatment site 302 while the neuromodulation device 112 is bioabsorbing. The period over which the neuromodulation device 112 disintegrates can be made to extend over any suitable time window (e.g., 4 hours, 24 hours, 1 week, 1 month, etc.) depending on the properties of the neuromodulation device 112, such as the type of bioabsorbable material used in the neuromodulation device 112 and the thickness of such material. In at least some cases, disintegrating the neuromodulation device 112 is thermally induced during neuromodulation, thereby expediting the disintegration and/or reducing or eliminating the possibility of premature disintegration.

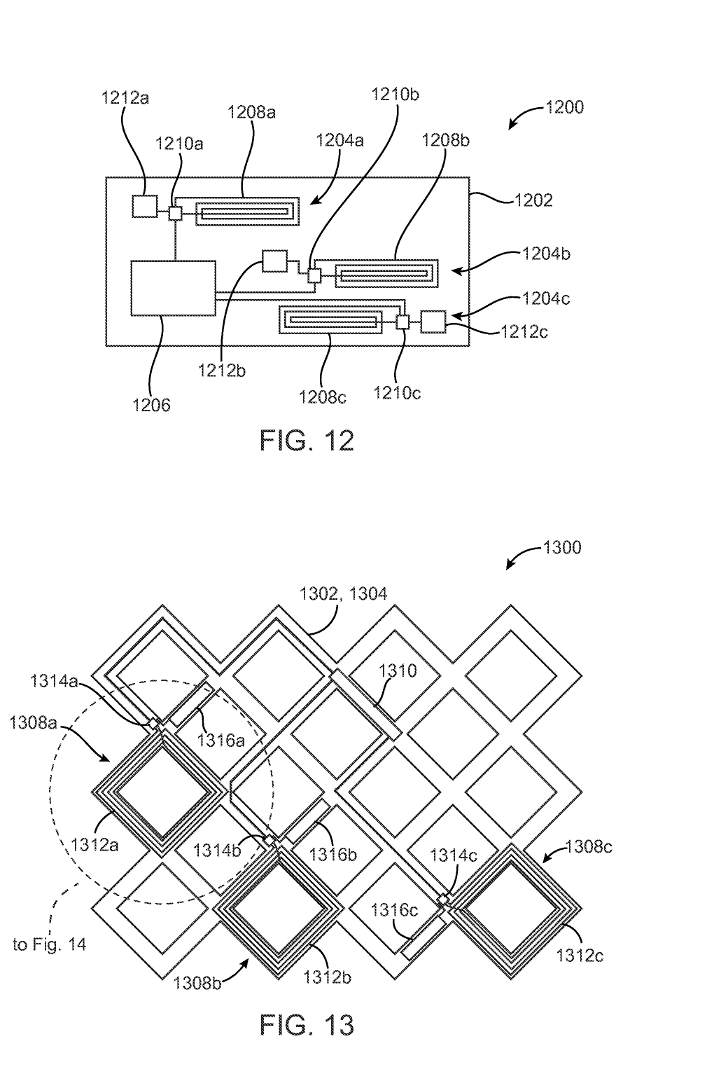

FIG. 12 is a flattened plan view of a neuromodulation device 1200 in accordance with another embodiment of the present technology. As shown in FIG. 12, the neuromodulation device 1200 can include a bioabsorbable membrane 1202 as a support structure. The membrane 1202 can be made at least partially (e.g., primarily) of a suitable bioabsorbable polymer, such as polylactic acid, polyglycolic acid, or a combination thereof. The neuromodulation device 1200 can further include a plurality of modules 1204 (individually identified as modules 1204a-1204c) and a controller 1206 operably connected to the modules 1204. The modules 1204 can respectively include inductors 1208 (e.g., induction coils) (individually identified as inductors 1208a-1208c), circuitry units 1210 (individually identified as circuitry units 1210a-1210c), and energy-delivery elements 1212 (e.g., electrodes, direct heat elements or ultrasound transducers) (individually identified as energy-delivery elements 1212a-1212c) operably coupled to one another. The individual circuitry units 1210 can include one or more capacitors (not shown), one or more switches (also not shown), and/or other suitable electrical components for supporting operation of the respective energy-delivery elements 1212. For example, the individual circuitry units 1210 can be configured to receive electricity from the respective inductors 1208 and to energize the respective energy-delivery elements 1212 using the received electricity in response to one or more signals from the controller 1206.

The controller 1206, the inductors 1208, the circuitry units 1210, and/or the energy-delivery elements 1212 can be bioresorbable. For example, an electrically conductive bioresorbable material can be printed or otherwise disposed onto the membrane 1202 to form one or more of these components. In one example, 5,5′-bis-(7-dodecyl-9H-fluoren-2-yl)-2,2′-bithiophene transistors are formed on a poly(vinyl alcohol) dielectric with a poly (L-lactide-co-glycolide) substrate. This example and others are described in Christopher J. Bettinger and Zhenan Bao, Organic Thin-Film Transistors Fabricated on Resorbable Biomaterial Substrates, 22 Adv. Mater. 651-655 (2010), which is incorporated herein by reference in its entirety. In another example, magnesium conductors, magnesium oxide dielectrics, and monocrystalline silicon nanomembrane semiconductors are disposed on a silk substrate to form bioresorbable electronics. This example and others are described in Suk-Won Hwang et al., A Physically Transient Form of Silicon Electronics, 337 Science 1640 (2012), which is incorporated herein by reference in its entirety.

In the illustrated embodiment, the neuromodulation device 1200 includes three energy-delivery elements 1212 arranged diagonally along a surface of the membrane 1202. When the neuromodulation device 1200 is curled into a cylindrical shape, the energy-delivery elements 1212 can be arranged in a helical shape well suited for forming lesions that are not circumferentially continuous in any single plane perpendicular to the axis of a vessel being treated, at least at the wall of the vessel. This can reduce or eliminate the possibility of the treatment causing stenosis of the vessel. In other embodiments, a greater or smaller number of energy-delivery elements 1212 can be used in the same or a different arrangement. Furthermore, rather than including separate inductors 1208 for the respective, energy-delivery elements 1212, one or more shared inductors 1208 can supply energy to multiple energy-delivery elements 1212. For example, a single inductor 1208 can supply energy to all energy-delivery elements 1212 of a neuromodulation device in accordance with a particular embodiment of the present technology.

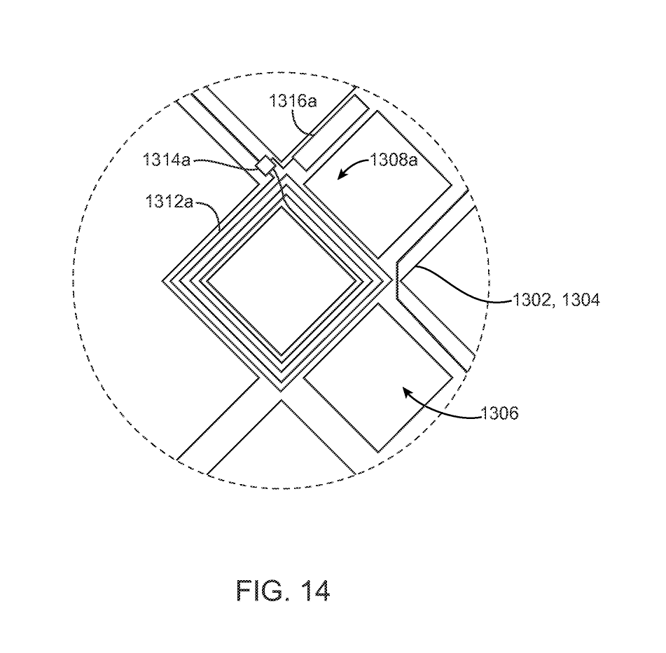

FIG. 13 is a flattened plan view of a neuromodulation device 1300 in accordance with another embodiment of the present technology. FIG. 14 is an enlargement of a designated portion of FIG. 13. With reference to FIGS. 13 and 14 together, the neuromodulation device 1300 can include a bioabsorbable scaffold 1302 as a support structure. Similar to the membrane 1202 discussed above, the scaffold 1302 can be made at least partially (e.g., primarily) of a suitable bioabsorbable polymer, such as polylactic acid, polyglycolic acid, or a combination thereof. The scaffold 1302 can include a network of struts 1304 and interstices 1306 between the struts 1304. The neuromodulation device 1300 can further include a plurality of modules 1308 (individually identified as modules 1308a-1308c) and a controller 1310 operably connected to the modules 1308. The modules 1308 can respectively include inductors 1312 (e.g., induction coils) (individually identified as inductors 1312a-1312c), circuitry units 1314 (individually identified as circuitry units 1314a-1314c), and energy-delivery elements 1316 (e.g., electrodes, direct heat elements or ultrasound transducers) (individually identified as energy-delivery elements 1316a-1316c) disposed along the struts 1304 and operably coupled to one another. The individual circuitry units 1314 can include one or more capacitors (not shown), one or more switches (also not shown), and/or other suitable electrical components for supporting operation of the respective energy-delivery elements 1316. For example, the individual circuitry units 1314 can be configured to receive electricity from the respective inductors 1312 and to energize the respective energy-delivery elements 1316 using the received electricity in response to one or more signals from the controller 1310.

With reference to FIGS. 12-14, in some cases, the neuromodulation devices 1200, 1300 are resiliently biased and configured to break apart to expand in transverse cross-sectional area. In other cases, the neuromodulation devices 1200, 1300 can be configured to retain respective tubular forms when expanded in transverse cross-sectional area. For example, the membrane 1202 can be configured to non-resiliently stretch as the neuromodulation device 1200 transitions from a delivery state to a treatment state. As another example, the interstices 1306 of the scaffold 1302 can be at least partially collapsed when the neuromodulation device 1300 is in a delivery state and can become enlarged as the neuromodulation device 1300 transitions into a treatment state. The controllers 1206, 1310, the inductors 1208, 1312, the circuitry units 1210, 1314, and/or the energy-delivery elements 1212, 1316 can be sufficiently flexible to accommodate these and/or other forms of expansion of the membrane 1202 and/or the scaffold 1302. Alternatively, controllers 1206, 1310, the inductors 1208, 1312, the circuitry units 1210, 1314, and the energy-delivery elements 1212, 1316 can be situated at portions of the membrane 1202 and/or the scaffold 1302 that do not expand.

### Renal Neuromodulation

Catheters configured in accordance with at least some embodiments of the present technology can be well suited (e.g., with respect to sizing, flexibility, operational characteristics, and/or other attributes) for performing renal neuromodulation in human patients. Renal neuromodulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically-induced, thermally-induced, or induced in another suitable manner or combination of manners at one or more suitable treatment sites during a neuromodulation procedure. The treatment site can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen. For example, with regard to a renal artery, a neuromodulation procedure can include modulating nerves in the renal plexus, which lay intimately within or adjacent to the adventitia of the renal artery. Various suitable modifications can be made to the catheters described above to accommodate different treatment modalities.

Renal neuromodulation can include an electrode-based or treatment modality alone or in combination with another treatment modality. Electrode-based or transducer-based treatment can include delivering electricity and/or another form of energy to tissue at or near a treatment site to stimulate and/or heat the tissue in a manner that modulates neural function. For example, sufficiently stimulating and/or heating at least a portion of a sympathetic renal nerve can slow or potentially block conduction of neural signals to produce a prolonged or permanent reduction in renal sympathetic activity. A variety of suitable types of energy can be used to stimulate and/or heat tissue at or near a treatment site. For example, neuromodulation in accordance with embodiments of the present technology can include delivering RF energy, pulsed electrical energy, microwave energy, optical energy, focused ultrasound energy (e.g., high-intensity focused ultrasound energy), and/or another suitable type of energy. An electrode or transducer used to deliver this energy can be used alone or with other electrodes or transducers in a multi-electrode or multi-transducer array.

Neuromodulation using focused ultrasound energy (e.g., high-intensity focused ultrasound energy) can be beneficial relative to neuromodulation using other treatment modalities. Focused ultrasound is an example of a transducer-based treatment modality that can be delivered from outside the body. Focused ultrasound treatment can be performed in close association with imaging (e.g., magnetic resonance, computed tomography, fluoroscopy, ultrasound (e.g., intravascular or intraluminal), optical coherence tomography, or another suitable imaging modality). For example, imaging can be used to identify an anatomical position of a treatment site (e.g., as a set of coordinates relative to a reference point). The coordinates can then entered into a focused ultrasound device configured to change the power, angle, phase, or other suitable parameters to generate an ultrasound focal zone at the location corresponding to the coordinates. The focal zone can be small enough to localize therapeutically-effective heating at the treatment site while partially or fully avoiding potentially harmful disruption of nearby structures. To generate the focal zone, the ultrasound device can be configured to pass ultrasound energy through a lens, and/or the ultrasound energy can be generated by a curved transducer or by multiple transducers in a phased array, which can be curved or straight.

Heating effects of electrode-based or transducer-based treatment can include ablation and/or non-ablative alteration or damage (e.g., via sustained heating and/or resistive heating). For example, a neuromodulation procedure can include raising the temperature of target neural fibers to a target temperature above a first threshold to achieve non-ablative alteration, or above a second, higher threshold to achieve ablation. The target temperature can be higher than about body temperature (e.g., about 37° C.) but less than about 45° C. for non-ablative alteration, and the target temperature can be higher than about 45° C. for ablation. Heating tissue to a temperature between about body temperature and about 45° C. can induce non-ablative alteration, for example, via moderate heating of target neural fibers or of luminal structures that perfuse the target neural fibers. In cases where luminal structures are affected, the target neural fibers can be denied perfusion resulting in necrosis of the neural tissue. Heating tissue to a target temperature higher than about 45° C. (e.g., higher than about 60° C.) can induce ablation, for example, via substantial heating of target neural fibers or of luminal structures that perfuse the target fibers. In some patients, it can be desirable to heat tissue to temperatures that are sufficient to ablate the target neural fibers or the luminal structures, but that are less than about 90° C. (e.g., less than about 85° C., less than about 80° C., or less than about 75° C.).

### CONCLUSION

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown or described in detail to avoid unnecessarily obscuring the description of the embodiments of the present technology. Although steps of methods may be presented herein in a particular order, in alternative embodiments the steps may have another suitable order. Similarly, certain aspects of the present technology disclosed in the context of particular embodiments can be combined or eliminated in other embodiments. Furthermore, while advantages associated with certain embodiments may have been disclosed in the context of those embodiments, other embodiments may also exhibit such advantages, and not all embodiments need necessarily exhibit such advantages or other advantages disclosed herein to fall within the scope of the present technology. Accordingly, this disclosure and associated technology can encompass other embodiments not expressly shown and/or described herein.

The methods disclosed herein include and encompass, in addition to methods of practicing the present technology (e.g., methods of making and using the disclosed devices and systems), methods of instructing others to practice the present technology. For example, a method in accordance with a particular embodiment of the present technology includes advancing an elongate shaft of a catheter toward a treatment site within a naturally occurring lumen of a human patient, deploying the neuromodulation device into an expanded treatment state at the treatment site, reducing obstruction of blood flow through the lumen at the treatment site, and modulating a nerve within tissue at or otherwise proximate to a wall of the lumen. A method in accordance with another embodiment of the present technology includes instructing such a method.

The headings provided herein are for convenience only and should not be construed as limiting the subject matter disclosed. As used herein, the terms “distal” and “proximal” define a position or direction with respect to an operator or an operator's control device (e.g., a handle of a catheter). The terms “distal” and “distally” refer to a position distant from or in a direction away from a clinician or a clinician's control device. The terms “proximal” and “proximally” refer to a position near or in a direction toward a clinician or a clinician's control device. Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various elements. It should be understood that such terms do not denote absolute orientation.

Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments of the present technology.

I/We claim:

1. A neuromodulation method, comprising:
advancing a neuromodulation device of a catheter toward a treatment site within a naturally occurring lumen of a human patient while the neuromodulation device is in a low-profile delivery state;
deploying the neuromodulation device into an expanded treatment state at the treatment site, blood flow through the lumen at the treatment site being at least partially obstructed while deploying the neuromodulation device;
reducing obstruction of blood flow through the lumen at the treatment site after deploying the neuromodulation device and while the neuromodulation device remains in the treatment state at the treatment site; and
modulating a nerve within tissue at or otherwise proximate to a wall of the lumen at the treatment site by delivering energy to the tissue via an energy-delivery element of the neuromodulation device while obstruction of blood flow through the lumen is reduced.

2. The method of claim 1 wherein the treatment site is within a renal artery of the patient.

3. The method of claim 1 wherein reducing obstruction of blood flow through the lumen at the treatment site includes reducing obstruction of blood flow through the lumen at the treatment site by at least 50%.

4. The method of claim 1 wherein the lumen has a diameter less than 2 millimeters at the treatment site.

5. The method of claim 1 wherein the neuromodulation device is advanced toward the treatment site while the neuromodulation device is constrained in the delivery state within a sheath.

6. The method of claim 5 wherein:
the neuromodulation device is deployed from within the sheath so as to allow the neuromodulation device to resiliently expand into the treatment state; and
obstruction of blood flow through the lumen at the treatment site is reduced by withdrawing the sheath away from the treatment site.

7. The method of claim 1 wherein:
modulating the nerve includes modulating the nerve a first time;
the method further comprises modulating the nerve a second time after modulating the nerve the first time by delivering energy to the tissue via the energy-delivery element while obstruction of blood flow through the lumen is reduced; and
the neuromodulation device remains in the treatment state at the treatment site for at least a portion of the time between the first and second nerve modulations.

8. The method of claim 7 wherein modulating the nerve the second time occurs at least 30 minutes after modulating the nerve the first time.

9. The method of claim 1, wherein the neuromodulation device disintegrates at the treatment site after the nerve has been modulated.

10. The method of claim 1, wherein the neuromodulation device disintegrates at the treatment site while the nerve is being modulated.

11. The method of claim 10, further comprising thermally inducing disintegration of the neuromodulation device at the treatment site while the nerve is being modulated.

12. The method of claim 1, further comprising recovering the neuromodulation device from the treatment site after modulating the nerve.

13. The method of claim 12 wherein:
the neuromodulation device is tubular and defines an interior region; and
recovering the neuromodulation device includes—
securing the neuromodulation device to a collapsible structure positioned within the interior region, and
decreasing a transverse cross-sectional area of the collapsible structure after securing the neuromodulation device to the collapsible structure.

14. The method of claim 13 wherein securing the neuromodulation device to the collapsible structure includes magnetically securing the neuromodulation device to the collapsible structure.

15. The method of claim 1, further comprising fully separating the neuromodulation device from a shaft of the catheter after deploying the neuromodulation device and while the neuromodulation device remains in the treatment state at the treatment site.

16. The method of claim 15 wherein:
the energy-delivery element includes an electrode; and
delivering energy to the tissue includes wirelessly energizing the electrode from an extracorporeal energy source.

17. The method of claim 16 wherein wirelessly energizing the electrode includes wirelessly energizing the electrode by resonant inductive coupling.

18. The method of claim 1 wherein:
the neuromodulation device is tubular and defines an interior region; and
deploying the neuromodulation device includes increasing a transverse cross-sectional area of an expandable structure within the interior region.

19. The method of claim 18 wherein reducing obstruction of blood flow through the lumen at the treatment site includes decreasing the transverse cross-sectional area of the expandable structure.

20. The method of claim 19 wherein the nerve is modulated while the expandable structure remains within the interior region.

21. The method of claim 19, further comprising withdrawing the expandable structure from the interior region before or while modulating the nerve.

22. An implantable neuromodulation device, comprising:
an energy-delivery element electrically energizable to modulate a nerve within tissue at or otherwise proximate to a wall of a naturally occurring lumen of a human patient; and
an elongate support structure carrying the energy-delivery element, the support structure being bioabsorbable,
wherein the support structure is configured to expand in a direction perpendicular to its length so as to move the energy-delivery element into contact with the wall of the lumen.

23. The device of claim 22 wherein the energy-delivery element includes a bioabsorbable electrode.

24. The device of claim 22, further comprising an inductor operably connected to the energy-delivery element.

25. The device of claim 22 wherein the support structure includes a membrane made at least primarily of a bioabsorbable polymer.

26. The device of claim 22 wherein the support structure includes a scaffold made at least primarily of a bioabsorbable polymer.

27. A neuromodulation system, comprising:
a catheter including an implantable neuromodulation device operably connected to an elongate shaft, the shaft being configured to locate the neuromodulation device at a treatment site within a naturally occurring lumen of a human patient, the neuromodulation device including—
an electrode activatable to modulate a nerve within tissue at or otherwise proximate to a wall of the lumen,
an elongate support structure carrying the electrode, the support structure being configured to expand in a direction perpendicular to its length within the lumen so as to move the electrode into contact with the wall of the lumen, and
an inductor operably connected to the electrode; and
an extracorporeal energy source configured to wirelessly energize the electrode.

28. The system of claim 27 wherein:
the inductor includes a first induction coil;
the extracorporeal energy source includes a second induction coil; and
the first and second induction coils are resonance coupled.

29. The system of claim 27 wherein the support structure is configured to break apart when it expands.

30. The system of claim 29 wherein:
the support structure includes a perforated seam; and
the support structure is configured to break apart at the seam when it expands.

31. The system of claim 27 wherein the catheter includes an expandable structure releasably carrying the neuromodulation device, the expandable structure being configured to expand so as to cause the support structure to expand.

32. The system of claim 31 wherein the expandable structure is a balloon.

33. The system of claim 31 wherein the expandable structure and the neuromodulation device are magnetically coupled to one another.
